(19) United States
(12) Patent Application Publication
(10) Pub. No.: US 2014/0274649 A1
(43) Pub. Date: Sep. 18, 2014

(54) Bone Marrow Concentrator

(71) Applicants: Sean Kerr, West Chester, PA (US); Jay Smith, West Chester, PA (US); Meredith Hans Moore, West Chester, PA (US)

(72) Inventors: Sean Kerr, West Chester, PA (US); Jay Smith, West Chester, PA (US); Meredith Hans Moore, West Chester, PA (US)

(21) Appl. No.: 13/826,332

(22) Filed: Mar. 14, 2013

(51) Int. Cl. A61M 1/36; B04B 7/12

(52) U.S. Cl. CPC A61M 1/3693; B04B 7/12
USPC 494/20; 494/37

(57) ABSTRACT

Instrumentation is provided for the separation of a multiple component sample, such as BMA containing lysed red blood cells and a lysing agent, into a desired component, for example a cell pellet containing stem cells, and a remaining component. The application discloses a device that includes a separator configured to separate the desired portion from the remaining portion, a collector that is supported by the separator and configured to collect the desired component of the multiple component sample after the desired component has been separated from the remaining component by the separator, and a housing that at least partially encloses and supports the separator and the collector.

### TECHNICAL FIELD

The present disclosure relates to a multiple component sample concentrator/separator. More particularly, the present disclosure relates to a multi-lobed centrifuge configured to separate and concentrate various biological components.

### BACKGROUND

Bone marrow aspiration involves inserting a needle into bone and withdrawing a material from the bone. The withdrawn material, for instance withdrawn bone marrow aspirate or “BMA,” can contain multiple components including plasma, red blood cells, and a buffy coat layer (that includes stem cells). After withdrawal of the multiple component sample the multiple components are often mixed together such that collection of a concentrated sample of any single component can be difficult. The multiple component sample can be separated into various components including, for instance a desired component (such as the buffy coat) and a remaining component (such as the plasma and red blood cells).

One process that can be used to separate the desired component from the remaining component of the multiple component sample is centrifugation. During centrifugation of the multiple component sample, for instance within a centrifuge device, each of the multiple components in the sample will assume a particular radial position within the device based upon the respective densities of each of the components. The multiple components will therefore separate when the centrifuge device is rotated at an appropriate angular velocity for an appropriate period of time.

Referring to FIG. 1A, a sample of withdrawn BMA 1 can be collected and prevented from clotting by the addition of an appropriate anticoagulant. The withdrawn BMA 1 can then be separated into its multiple component parts by a centrifuge 8. Centrifugation (or rotation about an axis of rotation 10) of the withdrawn BMA 1 will result in red blood cells 3, which are the densest part of the withdrawn BMA 1, being concentrated farthest from the axis of rotation 10 of the centrifuge 8 relative to the other parts of the withdrawn BMA 1. Plasma 7 (the least dense part of the withdrawn BMA 1) will be disposed nearest the axis of rotation 10 after centrifugation. The buffy coat 5 is located between the plasma 7 and the red blood cells 3.

Due to the intermediate position of the buffy coat 5 between the red blood cells 3 and the plasma 7 and also due to the relatively small size of the buffy coat layer 5 relative to the red blood cell layer 3 and the plasma layer 7, extraction of a concentrated volume of the buffy coat 5 after centrifugation can be difficult. One means of eliminating the red blood cell layer 3 is by lysing the red blood cells. A centrifugation device that enables the recovery of a high percentage of the desired component at a high concentration could result in time and cost savings for certain procedures.

### SUMMARY

The present disclosure provides, in accordance with one embodiment, a collection tray configured to rotate about an axis of rotation to separate a multiple component sample into a desired component and a remaining component. The collection tray can include a ray line that extends perpendicularly from the axis of rotation. The collection tray can include a collection body configured to receive the multiple component sample, and a plurality of lobes supported by the collection body. Each of the lobes can have two lobe base portions, an apex, and two lobe side walls that each extend between one of the lobe base portions and the apex. At least one of the lobes can define a straight lobe line that perpendicularly intersects one of the lobe side walls at a point located radially between the respective lobe base portion and the apex, such that the ray line intersects the point so as to define a lobe angle measured between the ray line and the lobe line. The lobe angle of the collection tray is greater than a specific angle, such that the arctangent of the specific angle is equal to the effective coefficient of friction of the desired component and the lobe side wall.

In accordance with another embodiment, the present disclosure provides a device configured to separate a multiple component sample into a desired component and a remaining component. The device includes a bowl portion defining an interior configured to receive the multiple component sample, and the bowl portion is configured to rotate about an axis of rotation. The device further includes a collection tray configured to be supported by the bowl portion so as to rotate about the axis of rotation. The collection tray defines a ray line that extends perpendicularly from the axis of rotation, and the collection tray includes at least one lobe that has two lobe base portions, an apex, and two lobe side walls that each extend from one of the lobe base portions to the apex. The at least one lobe at least partially defines a basin that is in fluid communication with the interior of the bowl portion such that the multiple component sample is transferable from the interior to the basin during rotation of the bowl portion about the axis of rotation. The at least one lobe further defines a lobe line that is different from the ray line, and the ray line intersects one of the lobe side walls at a point along the lobe side wall. The lobe line perpendicularly intersects the point so as to define a lobe angle between the ray line and the lobe line.

In accordance with another embodiment, the present disclosure provides a process to process a withdrawn BMA sample. The process includes the steps of: combining the withdrawn BMA sample and a red blood cell lysing agent so as to form a multiple component sample; rotating a device about an axis of rotation, the device containing the multiple component sample, so as to separate the multiple component sample into a desired component and a remaining component; and collecting at least a portion of the desired component.

### BRIEF DESCRIPTION OF THE DRAWINGS

The foregoing summary, as well as the following detailed description of the preferred embodiments of the application, will be better understood when read in conjunction with the appended drawings. For the purposes of illustrating the surgical instruments and methods of the present application, there is shown in the drawings preferred embodiments. It should be understood, however, that the application is not limited to the specific embodiments and methods disclosed, and reference is made to the claims for that purpose. In the drawings:

FIG. 1A is a side view of a centrifuge containing a multiple component sample;

FIG. 1B is a side view of the centrifuge illustrated in FIG. 1A containing a sample of BMA with the red blood cells lysed;

FIG. 2 is a cross-sectional view of a device according to one embodiment, the device including a separator, a collector, and a housing;

FIG. 3A is a cross-sectional schematic view of a portion of the separator illustrated in FIG. 2, the portion of the separator including a bowl portion, a collection tray and an axis of rotation;

FIG. 3B is a top plan schematic view portion of the separator illustrated in FIG. 3A;

FIG. 3C is a schematic top plan view of the portion of the separator illustrated in FIG. 3A, according to one embodiment;

FIG. 3D is a schematic top plan view of the portion of the separator illustrated in FIG. 3A, according to another embodiment;

FIG. 3E is a schematic top plan view of the portion of the separator illustrated in FIG. 3A, according to another embodiment;

FIG. 4A is a cross-sectional schematic view of the bowl portion, the collection tray, and the axis of rotation illustrated in FIG. 3A, after the bowl portion has been loaded with a multiple component sample and prior to rotation of the bowl portion and the collection tray about the axis of rotation;

FIG. 4B is a cross-sectional schematic view of the bowl portion and the collection tray illustrated in FIG. 3A, after the bowl portion has been loaded with the multiple component sample and during rotation of the bowl portion and the collection tray about the axis of rotation;

FIG. 5A is a top plan view of another portion of the separator illustrated in FIG. 2, the portion including a lid;

FIG. 5B is a top plan view of the lid illustrated in FIG. 2, according to another embodiment;

FIG. 5C is a cross-sectional view of the separator illustrated in FIG. 2, the separator including the bowl portion, the collection tray, and the lid in an assembled configuration;

FIG. 5D is a magnified cross-sectional view of the collection tray and the lid illustrated in FIG. 5C, the collection tray including a second locating feature and the lid including a first locating feature according to one embodiment;

FIG. 5E is a magnified cross-sectional view of the collection tray and the lid illustrated in FIG. 5C, the collection tray including a second locating feature and the lid including a first locating feature according to another embodiment;

FIG. 5F is a magnified cross-sectional view of the collection tray and the lid illustrated in FIG. 5C, the collection tray including a second locating feature and the lid including a first locating feature according to another embodiment;

FIG. 6A is a cross-sectional schematic view of the separator illustrated in FIG. 2, after the separator has been loaded with a multiple component sample and prior to rotation of the separator about the axis of rotation;

FIG. 6B is a top plan view of the separator illustrated in FIG. 6A, after the separator has been loaded with a multiple component sample and prior to rotation of the separator about the axis of rotation;

FIG. 6C is a cross-sectional schematic view of the separator illustrated in FIG. 6B, after the separator has been loaded with a multiple component sample and after rotation of the separator about the axis of rotation has commenced;

FIG. 6D is a cross-sectional schematic view of the separator illustrated in FIG. 6C, after the separator has been loaded with a multiple component sample and during additional rotation of the separator about the axis of rotation;

FIG. 6E is a cross-sectional schematic view of the separator illustrated in FIG. 6D, after the separator has been loaded with a multiple component sample and after rotation of the separator about the axis of rotation has been completed;

FIG. 6F is a top plan view of the separator illustrated in FIG. 6A, after the separator has been loaded with a multiple component sample and after to rotation of the separator about the axis of rotation has been completed;

FIG. 7A is a top plan view of the collector illustrated in FIG. 2 according to one embodiment, in a first retracted configuration;

FIG. 7B is a top pan view of the collector illustrated in FIG. 7A, in a second expanded configuration;

FIG. 7C is a perspective view of the collector illustrated in FIG. 7A, in the first retracted configuration;

FIG. 7D is a top plan view of the collector illustrated in FIG. 2 according to another embodiment, in the second expanded configuration;

FIG. 8A is a cross-sectional schematic view of the device illustrated in FIG. 2 after rotation of the separator about the axis of rotation has been completed, wherein the collector is secured relative to the separator according to one embodiment, and the collector is in the first retracted configuration;

FIG. 8B is a cross-sectional schematic view of the device illustrated in FIG. 8A, wherein the collector in the second expanded configuration;

FIG. 8C is a magnified cross-sectional schematic view of a portion of the device illustrated in FIG. 8A, wherein the collector is secured relative to the separator according to another embodiment;

FIG. 8D is a top plan view of the collector secured to the separator as illustrated in FIG. 8C;

FIG. 9 is a top plan view of the collector according to another embodiment;

FIG. 10 is a top plan view of the collector illustrated in FIG. 7A according to one embodiment, with the collector is in the first retracted configuration;

FIG. 11A is a top plan view of the collector illustrated in FIG. 7A according to another embodiment, with the collector is in the first retracted configuration;

FIG. 11B is a top plan view of the collector illustrated in FIG. 11A, the collector being transitioned from the first retracted configuration to the second expanded configuration;

FIG. 11C is a top plan view of the collector illustrated in FIG. 11A, the collector being transitioned from the first retracted configuration to the second expanded configuration;

FIG. 11D is a top plan view of the collector illustrated in FIG. 11A, the collector in the second expanded configuration;

FIG. 12 is a cross sectional view of the housing illustrated in FIG. 2, the housing including an outer shell and a cap.

### DETAILED DESCRIPTION OF THE ILLUSTRATIVE EMBODIMENTS

Certain terminology is used in the following description for convenience only and is not limiting. The words “upper”, “lower”, “above” and “below” designate directions in the drawings to which reference is made. The terminology includes the above-listed words, derivatives thereof and words of similar import. Additionally, a radial or polar coordinate system is provided and described herein. The polar coordinate system includes a two dimensional radial plane that is centered on and normal to an axis, for instance an axis of rotation. The polar coordinate system defines a radial component that is measured as the distance from the axis along the plane. The words “inner” and “outer” designate locations closer to and farther away from the axis respectively. The polar coordinate system further defines an angular component that is measured as the angular position about the axis. The radial coordinate system can be converted to a three dimensional coordinate system, for instance a right-hand coordinate system that includes a first or longitudinal direction L, a second or lateral direction A that is perpendicular to the longitudinal direction L, and a third or transverse direction T that is perpendicular to both the longitudinal direction L and the lateral direction A. The longitudinal direction L and the lateral direction A can define a plane that corresponds to the radial plane and position along the radial axis corresponds to position in the transverse direction T.

The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. Further, reference to values stated in ranges includes each and every value within that range. All ranges are inclusive and combinable. Certain features of the invention which are described herein in the context of separate embodiments, may also be provided in combination in a single embodiment. Conversely, various features of the invention that are described in the context of a single embodiment, may also be provided separately or in any subcombination.

Referring to FIGS. 1A and 1B, the addition of a hypotonic solution, for instance 0.5 percent ammonium chloride, to the withdrawn BMA sample 1 will result in lysing of the red blood cells 3. The lysing agent ruptures the red blood cell membranes, resulting in a supernatant layer 13 (which can contain the contents of the lysed blood cells 3, the lysing agent, and the plasma 7) and a buffy coat layer 5. Because the buffy coat 5 is denser than the supernatant 13, after the lysed BMA 11 has undergone centrifugation, the buffy coat 5 will be concentrated farther from the axis of rotation 10 of the centrifuge 8 relative to the supernatant 13 to form a cell pellet 15. This positioning of the cell pellet 15 at the outer most periphery of the centrifuge 8 can result in more efficient collection of a concentrated amount of the cell pellet 15 as will be described in greater detail below.

Referring to FIGS. 1A to 2, a multiple component handling device 18 (hereinafter referred to as “the device”) can include a separator apparatus 20 (hereinafter referred to as “the separator”) configured to separate the components of a multiple component sample, for instance the multiple component sample can be withdrawn and lysed BMA 11. The separator 20 can be used to separate the lysed BMA 11, for instance by centrifugation, to result in lysed and centrifuged BMA 11. The lysed and centrifuged BMA 11 can include a desired component, for instance a cell pellet 15, and a remaining component, for instance a supernatant layer 13 including red blood cells 3 that have been lysed, lysing agent, and plasma 7. The separator 20 can be configured to separate the desired component from the remaining component such that a concentrated sample of the desired component can be collected. The device 18 can further include a collection apparatus 100 (hereinafter referred to as “the collector”) that is secured relative to the separator 20 and configured to collect the desired component of the multiple component sample after the desired component has been separated from the remaining component by the separator 20. The device 18 can also include a housing 300 that at least partially encloses and supports the separator 20 and the collector 100.

As will be described in greater detail below, the device 18 can be configured such that the desired component, for example the cell pellet 15, of the lysed and centrifuged BMA 1, has a volumetric concentration of stem cells, that is greater than the average volumetric concentration of stem cells of the withdrawn BMA 1 prior to lysing and centrifugation. In accordance with one embodiment, the volumetric concentration of stem cells in the cell pellet 15 can be at least a multiple, such as four fold, of the average volumetric concentration of stem cells in the withdrawn BMA 1. In one embodiment, the device 18 can be configured such that the cells of the desired component, for instance the stem cells of the cell pellet 15, maintain at least 95% viability during separation and collection by the device 18. In one embodiment, the device 18 is configured to complete the separation and collection of the desired component from the remaining component in 30 minutes or less, such that the device 18 can be used intraoperatively.

The device 18 can be configured to accept a range of volumes of withdrawn BMA 1. The volume of withdrawn BMA 1 can be separated into the desired component and the remaining component and the desired component can then be collected. In one embodiment, the device 18 is configured to accept and separate any volume of withdrawn BMA 1 as desired, for example between about 8 cc to about 50 cc. Furthermore, it should be appreciated that the withdrawn BMA 1 can be lysed either prior to introduction into the device 18 or after introduction to the device 18. In one embodiment, the device 18 is configured to be ergonomic and intuitive such that the device 18 is easy to use in an operating room environment. The device 18 can be configured such that the separator 20, collector 100, and the housing 300 can be double packaged and sterilized. The device 18 can also be configured to be disposable, such that after the separation and collection of the desired component of a multiple component sample, the device 18 can be thrown away. The device 18 can further be configured to provide maximum portability such that the device 18 is cordless or self-contained, for instance battery powered with no external power source needed.

Referring to FIG. 3A, the separator 20 includes an axis of rotation 22 and a container, for example a bowl portion 24 that is rotatable about the axis of rotation 22. The bowl portion 24 includes an inner surface 28, an outer surface 30 and a bowl body 32 that extends from the inner surface 28 to the outer surface 30. The bowl body 32 can include an engagement mechanism 33 that is configured to receive a rotational force that rotates the bowl portion 24 about the axis of rotation 22. As shown in the illustrated embodiment, the engagement mechanism 33 can include a post 35 that defines a recess 37, the recess 37 being configured to engage a rotating member, for instance a drive shaft, which imparts the rotational force to the bowl portion 24 that causes the bowl portion 24 to rotate about the axis of rotation 22.

The bowl body 32 includes a bowl bottom 34, an upper lip 36, and a height H1 measured from the bowl bottom 34 to the upper lip 36. The bowl body 32 further includes a bowl wall 38 that extends from the bowl bottom 34 to the upper lip 36 and an inner diameter D1 that is measured from one side of the bowl wall 38 to another side of the bowl wall 38 along a straight line that passes perpendicularly through the axis of rotation 22. The bowl wall 38 is angularly offset from the axis of rotation 22 such that the inner diameter D1 of the bowl body 32 gradually increases from a minimum value at the bowl bottom 34 to a maximum value at the upper lip 36. The bowl body 32 can be configured with a height H1 and an inner diameter D1 such that the bowl portion 24 can be filled with a range of volumes of multiple component sample and still produce effective separation of the desired component from the remaining component.

In one embodiment the height H1 and the inner diameter D1 of the bowl body 32 are configured such that the bowl portion 24 is capable of receiving any desired volume of withdrawn BMA 1, such as between about 8 cc to about 50 cc in addition to a volume of lysing agent. The amount of lysing agent can be, for example, twice the volume of the volume of withdrawn BMA 1. Thus for a volume of withdrawn BMA 1 between about 8 cc to about 50 cc, the volume of lysing agent can be between about 16 cc to about 100 cc. Thus, the dimensions of the bowl body 32, including the height H1 and the inner diameter D1 can be chosen from a range of values such that the bowl portion 24 is configured to receive a range of total volume of withdrawn BMA 1 and lysing agent between about 24 cc to about 150 cc.

Referring to FIGS. 3A and 3B, the separator 20 can further include a collection tray 26 that is supported by, for example rotationally secured to, the bowl portion 24, for example the upper lip 36 of the bowl body 32 such that the collection tray 26 and the bowl portion 24 are configured to rotate together about the axis of rotation 22. The collection tray 26 can include an inner rim 40 that coincides with the upper lip 36 of the bowl body 32. The collection tray 26 extends radially outward from the inner rim 40 to a tray outer periphery 42, and the collection tray 26 further includes a collection body 44 that extends from the inner rim 40 to the tray outer periphery 42. The collection body 44 includes lobes 46 that are each configured to receive and concentrate the desired component during rotation of the separator 20 about the axis of rotation 22 and to retain the desired component to be collected after rotation of the separator 20 about the axis of rotation 22 has completed. The lobes 46 can be spaced about the collection body 44 such that each of the lobes 46 extends from the inner rim 40 radially toward the outer periphery 42. Although the illustrated embodiment is shown with four lobes 46, it will appreciated that the collection body 44 can include any number of lobes 46, for example between about 2 to about 10 lobes. The lobes 46 can be arranged about the collection body 44 such that the bowl portion 24 is balanced and will spin smoothly without vibration.

Each of the lobes 46 includes two base portions 48 and an apex 50 disposed radially farther from the axis of rotation 22 than each of the two base portions 48. In another embodiment, the lobes 46 can include more than two base portions 48. Each of the lobes 46 further includes two lobe side walls 52 that each extends between one of the two base portions 48 and the apex 50. In one embodiment, the lobe side wall 52 is the radially outward most component of the collection body 44. Each of the lobe side walls 52 can include an inner side wall 53 and an outer side wall 55, the outer side wall 55 being disposed radially farther from the axis of rotation 22 than the inner side wall 53. In one embodiment, the outer side wall 55 is the radially outward most component of the collection body 44. Each of the lobes 46 can further include a floor 51 that extends at least partially radially in a first direction between the inner rim 40 and the apex 50 and angularly in another direction between the inner side wall 53 of each of the side walls 52 of the respective lobe 46. The inner side walls 53 of the two side walls 52 and the floor 51 together define a basin 57 of the lobe 46 that is configured to receive a volume of the multiple component sample during rotation of the separator 20 about the axis of rotation 22.

The lobes 46 can be configured such that the cumulative volume of all of the basins 57 of all of the lobes 46 is greater than or equal to the total volume of the desired component that will be separated from the remaining component after centrifugation of the multiple component sample. For example if a 50 cc sample of withdrawn BMA 1 is placed in the separator 20 along with a 100 cc sample of lysing agent, the desired component is a fraction, for instance one-twelfth or 12.5 cc of the total volume of lysed BMA 11. In this example, if the separator 20 includes a collection tray 26 with four lobes 46, each of the basins 57 of the four lobes 46 could be configured to define a volume of at least 3.2 cc. A variety of collection trays 26 can include a number of different configurations of lobes 46 with basins 57 that define various volumes to accommodate samples of various volumes and desired ratios of total sample to desired component.

In one embodiment, the side walls 52 define a midpoint 59 that is located radially halfway between the base portion 48 and the apex 50. The side walls 52 each include a proximal portion 61 located between the base portion 48 and the midpoint 59 and a distal portion 63 located between the midpoint 59 and the apex 50. The side walls 52 can be curved, for instance such that the inner side wall 53 is concave and the outer side wall 55 is convex, as shown in the illustrated embodiment. In one embodiment, the inner side wall 53 within the proximal portion 61 of lobe 46 is curved such that no portion of the inner side wall 53 is parallel to a radial ray 65 that extends straight out from the axis of rotation 22 and intersects the apex 50 of the respective lobe 46. In another embodiment, the inner side wall 53 is curved such that no portion of the inner side wall 53 extends purely radially (or only in the radial direction).

The lobes 46 each define a lobe angle β that is measured between a radial ray 54 (a straight line extending from and perpendicular to the axis of rotation 22 to a point on the inner side wall 53) and a lobe line 56 (a straight line that perpendicularly intersects the inner side wall 53 at the point). In one embodiment, the collection body 44 of the collection tray 26 is configured such that the lobe angle β has a desired value greater than a certain value (referred to herein as the “specific value”). The specific value of the lobe angle β is defined such that when a component of the sample, such as a mononucleated cell, is in contact with the inner side wall 53 and a radial force is applied to the component of the sample (such as centripetal force when the bowl portion 24 and the collection tray 26 are spinning, or rotating, about the axis of rotation 22), the component of the sample will move relative to the inner side wall 53. In one embodiment, the specific value of the lobe angle β can be determined by calculating the inverse tangent or the arctangent (TAN⁻¹) of the effective coefficient of friction of the desired component and the inner side wall 53. The calculation can be represented by the following equation: (specific value)=TAN⁻¹ (effective coefficient of friction).

For example, referring to FIGS. 3C to 3E, if the desired component of the multiple component sample has an effective coefficient of friction with the inner side wall 53 of about 0.09, the specific value for the lobe angle β would be about 5 degrees. Thus a separator 20 configured with a lobe angle β of about 5 degrees (as shown in FIG. 3C) or greater would allow the desired component to move along the inner side wall 53 during rotation of the separator 20. In another embodiment, if the desired component of the multiple component sample has an effective coefficient of friction with the inner side wall 53 of about 0.18, the specific value for the lobe angle β would be about 10 degrees. Thus a separator 20 configured with a lobe angle β of about 10 degrees (as shown in FIG. 3D) or greater would allow the desired component to slide along the inner side wall 53 during rotation of the separator 20. In another embodiment, if the desired component of the multiple component sample has an effective coefficient of friction with the inner side wall 53 of about 0.36, the specific value for the lobe angle β would be about 20 degrees. Thus a separator 20 configured with a lobe angle β of about 20 degrees (as shown in FIG. 3E) or greater would allow the desired component to move along the inner side wall 53 during rotation of the separator 20.

The material of the inner side wall 53, the surface smoothness of the inner side wall 53, and the constituents of the multiple component sample can all affect the effective coefficient of friction and therefore the specific value. In one embodiment a coating, can be applied to inner side wall 53 to change the effective coefficient of friction between the inner side wall 53 and the desired component. In one embodiment, PTFE (Teflon) coating can be applied to the inner side wall 53, for instance by spraying or a mechanical process. Once the specific value for the lobe angle β has been determined the separator 20 can be configured with a lobe angle β that is chosen to be greater than the specific value. The actual lobe angle β can be chosen based on additional factors related to ease of construction and operation, size restrictions, ease of collection, etc.

Referring again to FIGS. 3A and 3B, in one embodiment the collection tray 26 can be selected from a kit of multiple collection trays 26 with various lobe angles 3 based on the specific multiple component sample that is to be separated and the desired sample that is being collected. For example, if the multiple component sample being separated is lysed BMA 11, the lobe angle β could be from about 10 degrees to about 30 degrees, or more specifically from about 15 degrees to about 20 degrees. In another embodiment, the collection tray 26 can be selected with a lobe angle β based at least partially on the angular velocity used during centrifugation. For example, increasing the angular velocity of the centrifuge can result in a smaller lobe angle β being needed for the desired component to move along the inner side wall 53 during rotation of the separator 20.

In one embodiment, the lobe angle β can be substantially constant measured at any point along the side wall 52. As shown in the illustrated embodiment, the lobe angle β can be measured at a first point 52a near the base portion 48, at a second point 52b near the apex 50, or at a third point 52c nearly midway between the base portion 48 and the apex 50. In one embodiment, the lobe angle β is substantially the same at first point 52a, second point 52b, and third point 52c. In another embodiment, the lobe angle β can vary as measured at different points along the side wall 52. For example the lobe angle β measured at each of the first, second, and third points 52a, 52b and 52c, can be different, but always greater than the specific value.

The lobes 46 can further include an inner tray surface 58 and an opposed outer tray surface 60. As shown in the illustrated embodiment, the inner tray surface 58 can define a negative slope such that the inner tray surface 58 extends downward (in a direction from the inner rim 40 toward the bowl bottom 34 and parallel to the axis of rotation 22) and radially outward (in a direction from the inner rim 40 toward the tray outer periphery 42 and perpendicular to the axis of rotation 22).

The inner tray surface 58 defines a collection area, such as a pocket 62 that is configured to collect a concentrated sample of the densest component of the multiple component sample during rotation of the separator 20 about the axis of rotation 22. In one embodiment the pocket 62 is the radially most distant part of the basin 57. The negative slope of the inner tray surface 58 is configured such that when the bowl portion 24 stops rotating about the axis of rotation 22, the densest component of the multiple component sample, for instance the cell pellet 15 in a sample of lysed BMA 11, is retained in the pocket 62 for collection. In one embodiment the inner tray surface 58 defines a vertical offset 64 that is the distance between the pocket 62 and the inner rim 40 as measured along a direction parallel to the axis of rotation 22 (or in the transverse direction T). As shown in the illustrated embodiment, the vertical offset 64 can be configured such that a portion of the basin 57 is located below (or downward relative to) the inner rim 40. In one embodiment, the cumulative volume of the basin 57 of each of the lobes 46 that is located below the inner rim 40 is equal to or greater than the volume of the desired component of the multiple component sample.

Although the collection tray 26 and bowl portion 24 are shown as integral or monolithic parts in the illustrated embodiment, in another embodiment, the collection tray 26 can be a separate or separable part with respect to the body portion 24 such that a collection tray 26 with a desired lobe angle β can be chosen from a kit containing a plurality of collection trays 26 with a plurality of lobe angles β, based on the particular multiple component sample that is to be separated. In this embodiment the collection tray can be a monolithic body such that each of the lobes 46 are integral (or not easily separable) with one another. Once the collection tray 26 with the desired lobe angle β is chosen, the collection tray can be attached to the bowl portion 24.

As shown in FIG. 3A, the bowl body 32 further includes a bowl angle θ defined by a radial ray 27 (a line extending out from and perpendicular to the axis of rotation 22 to a point on the bowl wall 38) and a bowl line 29 (a line normal to the bowl wall 38 at the point). In one embodiment the bowl body can be configured such that the bowl wall angle θ is greater than or equal to a specific bowl wall value. In another embodiment, the specific bowl wall value can be determined by the following equation: (specific bowl wall value)=TAN−1 (effective coefficient of friction). For example, if the effective coefficient of friction between the bowl wall 38 and the desired component is about 0.28 the specific value would be about 15 degrees. In one embodiment, the specific bowl wall angle can be from about 10 degrees to about 40 degrees. Note that the specific bowl wall angle may be different than the specific value for the lobe angle, depending on material selection and surface smoothness. The actual bowl angle θ can be selected based on practical considerations including ease of manufacture and operation, cost effectiveness, etc.

Referring to FIGS. 4A and 4B, the bowl portion 24 contains a multiple component sample, for instance a sample of lysed BMA 11. The bowl angle θ is configured such that when the bowl portion 24 is rotated about the axis of rotation 22 the multiple component sample, including the densest component of the multiple component sample, will move radially away from the axis of rotation 22 and toward the bowl wall 38, and then move up the bowl wall 38 toward the upper lip 36. Upon reaching the upper lip 36 the multiple component sample passes over the upper lip 36 and into the collection tray 26 (as shown by the arrows).

Referring to FIGS. 5A to 5F, the separator 20 can further include a lid 70 that is configured to be supported by, for example secured or located relative to, the collection tray 26 such that during rotation of the bowl portion 24 and the lid 70 about the axis of rotation 22, the multiple component sample is retained within the separator 20 and prevented from splashing, spinning, or otherwise exiting the separator 20.

The lid 70, as shown in the illustrated embodiment can be centered on the axis of rotation 22 such that the lid 70 is configured to spin or rotate about the axis of rotation 22 when the lid 70 is secured to the collection tray 26. The lid 70 defines a lid outer periphery 72, and the lid 70 includes a lid body 74 that extends radially between the axis of rotation 22 and the lid outer periphery 72. The lid body 74 can include a lobe portion 76 and a dome portion 78. The lobe portion 76 can include lid lobes 80 that correspond (for example, in number and shape) to the lobes 46 of the collection body 44. The lobe portion 76 can further include a lid inner surface 81 that along with the inner tray surface 58 defines the pocket 62 when the lid body 74 is properly secured to the collection body 44.

Referring to FIGS. 5A to 5C, the dome portion 78 can include one or more openings 82 that are configured to both prevent or limit the multiple component sample from escaping the separator 20 during rotation about the axis of rotation 22, and permit the entry of a collection tool or collector into the pocket 62 to remove a concentrated sample of a desired component of the multiple component sample after rotation of the separator 20 about the axis or rotation 22 (and separation of the multiple component sample) has been completed. The one or more openings 82 can include a single aperture, such as circular aperture 84 shown in FIG. 5A, or multiple spaced apertures, such as elliptical apertures 86 shown in FIG. 5B. Alternatively, any number of apertures of any desired shape can be spaced about the lid body 74 such that a collection tool can access the pocket 62 of each of the lobes 46. In another embodiment, the openings 82 can be configured such that they can be partially closed or completely shut during rotation of the separator 20 about the axis of rotation 22 and opened during collection of a desired component of the multiple component sample.

Referring to FIGS. 5C to 5F, the lid body 74 can further include a first locating feature 88 that is configured to locate the lid body 74 to the collection body 44 during rotation of the bowl portion 24 and the lid 70 about the axis of rotation 22. In one embodiment the first locating feature 88 can include a lid outer side wall 90 that is configured to fit at least partially within the tray outer periphery 42. As shown in FIG. 5D, the lid outer side wall 90 can be configured to fit within a corresponding second locating feature 66 such that the lid body 74 is located to the collection body 44 during rotation of the bowl portion 24 and the lid 70. The first locating feature 88 and second locating feature 66 can include a corresponding projection 92 and recess 68. The projection 92 is configured to fit, within the recess 68.

In one embodiment, for instance as shown in FIG. 5E, the first locating feature 88 and the second locating feature 66 can be reversed relative to the previous embodiment of FIG. 5D such that the tray outer periphery 42 fits at least partially within the lid outer periphery 72, for instance the first locating feature 88 can include a recess 93 that is configured to receive a projection 69 of the second locating feature 66. As shown in FIG. 5F, in another embodiment the first and second locating features can include a tongue and groove mechanism. The first locating feature 88, in one embodiment, is a groove 95 that is configured to receive a tongue 71 of defined by the second locating feature 66. Alternatively, the tongue and groove mechanism could be reversed such that the first locating feature 88 defines the tongue and the second locating feature 66 defines the groove.

In another embodiment, the lid body 74 may be secured to the collection body 44 using an adhesive, which may also fill any potential gaps between the lid body 74 and the collection body 44.

Referring to FIGS. 6A to 6F, a multiple component sample, for instance a sample of lysed BMA 11, can be placed in the bowl portion 24 and the bowl portion 24, collection tray 26, and the lid 70 can be secured relative to one another in an assembled configuration. The assembled bowl portion 24, collection tray 26, and lid 70 can then be rotated about the axis of rotation 22 to separate the lysed BMA 11 into its multiple components. As shown in FIGS. 6A and 6B, the lysed BMA 11 has been placed in the bowl portion 24 of the separator 20. Prior to rotation of the bowl portion 24 about the axis of rotation 22, the multiple components of the lysed BMA 11 are fairly homogeneously mixed throughout the lysed BMA 11.

As the bowl portion 24 begins to rotate about the axis of rotation 22, as shown in FIG. 6C, the lysed BMA 11 begins to move radially away from the axis of rotation 22. The multiple components of the lysed BMA 11, the cell pellet 15 and the supernatant 13 begin to separate from each other. The densest component of the lysed BMA 11, for example the cell pellet 15 as shown in the illustrated embodiment, moves radially away from the axis of rotation 22 and toward the bowl wall 38, and then up the bowl wall 38 toward the upper lip 36. Upon reaching the upper lip 36 the cell pellet 15 passes over the upper lip 36 and into the collection tray 26. The cell pellet 15 then enters the basin 57 and then continues to move radially away from the axis of rotation 22 until the cell pellet reaches the pocket 62. The supernatant 13 also moves radially away from the axis of rotation 22 and toward the bowl wall 38, moves up toward the upper lip 36, passes over the upper lip 36 and into the collection tray 26, and then advances toward the pocket 62 forming a layer of supernatant 13. Because the supernatant 13 is less dense than the cell pellet 15, the supernatant 13 is generally disposed radially closer to the axis of rotation 22 than the cell pellet 15. The bowl portion 24 continues to rotate about the axis of rotation 22 until substantially all of the cell pellet 15 has been separated from the supernatant 13, and the cell pellet 15 has collected within the pocket 62 such that the cell pellet 15 is disposed at the most radially distant position from the axis of rotation 22 within the separator 20, as shown in FIG. 6D.

After substantially all of the cell pellet 15 has been separated from the supernatant 13 and concentrated in the pocket 62, rotation of the bowl portion 24 and the lid 70 about the axis of rotation 22 can be terminated. As shown in FIGS. 6E and 6F, once rotation of the separator 20 has ceased, the cell pellet 15 is collected within a portion of the pocket 62 that is located most radially distant from the axis of rotation 22. While a portion of the supernatant 13 can also remain within collection tray 26, the majority of the supernatant 13 settles back into the bowl portion 24 of the separator 20. This arrangement of the cell pellet 15 relative to the supernatant 13 enables the collection of a concentrated sample of the cell pellet 15.

Referring to FIGS. 7A to 7C, the device 18 can include a collector 100 that is configured to collect or retrieve a concentrated sample of a desired component of a multiple component sample, for instance the cell pellet 15 of a sample of lysed and centrifuged BMA 11. The collector 100 can include a housing 104 and a probe 102 supported by the housing 104. The probe 102 includes an attached end 106 that is configured to attach to the housing 104 such that the probe 102 is secured relative to the housing 104. The probe further includes a free end 108 that is opposite the attached end 106. The probe 102 can further include a probe body 105 extending from the attached end 106 to the free end 108, and a cannula 110 that extends through the probe body 105 from the free end 108 to the attached end 106.

The collector 100, as shown in the illustrated embodiment, can further include a collection container, for instance a syringe 118, that is configured to be supported by the housing 104, for example at an attachment point 125, and collect and contain an amount of a concentrated sample of a desired component of a multiple component sample. The collection container is connected to the free end 108 of the probe 102 such that the desired component collected by the probe 102 is transferred to the collection container. For example, the syringe 118 can be pneumatically connected to the free end 108 of the probe 102.

The collector 100 can further include a guide rod 112 with a first end 114 and a second end 116 opposite the first end 114. In one embodiment, the housing 104 is configured to translate along the guide rod 112 from a first contracted configuration (as shown in FIG. 7A) to a second expanded configuration (as shown in FIG. 7B). In the second expanded configuration the free end 108 of the probe 102 is spaced farther from the first end 114 of the guide rod 112 then when in the first contracted configuration.

The collector 100 can additionally include one or more scrapers 120, that are configured to aid in the collection a concentrated sample of a desired component of a multiple component sample. Each of the one or more scrapers 120 can be attached to the housing 104, for instance to a flange 121 of the housing 104 such that the probe 102, the housing 104, and the at least one scraper 120 are all translationally locked relative to each other, such that as the housing translates along the guide rod 112, for example in the radial or specifically in the longitudinal direction L, the probe 102 and the at least one scraper 120 also translate along with the housing 104 in the same direction as the housing 104. As shown in the illustrated embodiment, the collector 100 can include a body 103 that functions both as the scraper 120 and as the probe 102 are described within the present disclosure.

The collector 100 defines a passage 122 from the free end 108 of the probe 102 to the attachment point 125 of the syringe 118. The passage 122 provides a path for the collected sample to pass through the collector 100 from the free end 108 of the probe 102 to a receiving chamber 119 of the syringe 118. As shown in the illustrated embodiment, the probe 102 defines a cannula 110 (shown in dashed lines) that extends through the body 105 from the free end 108 to the attached end 106. The collector 100 can further include a tube 123 that is connected, for example pneumatically, to the attached end of the probe 102. In one embodiment the tube 123 at least partially defines the passage 122, and is pneumatically connected to the attachment point 125.

Once the collector 100 has been moved into the second expanded configuration such that the free end 108 of the probe 102 is positioned within the desired component of the multiple component sample, a plunger 128 of the syringe 118 can be actuated to draw the desired component into the passage 122 for collection. As the plunger 128 is actuated, the desired component adjacent the free end 108 of the probe is drawn into the cannula 110 of the probe 102 at the free end 108. The desired component is then drawn in a direction toward the attached end 106 of the probe 102 (or proximally). The desired component is next drawn into the tube 123 which connects the probe 102 to the syringe 118. It will be apparent to one of skill in the art that the arrangement and selection of the components of the collector 100 that form the passage 122, for example the tube 123, could be changed or substituted without deviating from the teachings of the present disclosure.

Referring to FIG. 7D, in another embodiment the collector 100 includes a probe 102 that is spaced apart or separate from the scrapers 120. The probe 102 can be in the form of a cannulated tube that is attached directly to the housing 104 such that as the housing 104 translates along the guide rod 112 from the first contracted configuration to the second expanded configuration, the free end 108 of the probe advances towards desired component.

Referring to FIGS. 8A and 8B, the collector 100 is configured to be positioned at least partially within the separator 20 such that the collector 100 can collect a sample of a desired component of a multiple component sample. As shown in the illustrated embodiment, the collector 100 is configured to attach to the housing 300. The separator 20 is rotatable relative to the housing 300 such that as the separator 20 rotates about the axis of rotation 22, the housing 300 does not rotate about the axis of rotation 22. In one embodiment, the collector 100 includes a bracket 130 that is configured to secure the collector 100 to the housing 300.

In one embodiment the bracket 130 includes an inner bore that is configured to receive the guide rod 112. Once the guide rod 112 has been received within bracket 130, the guide rod 112 can be secured relative to the bracket 130 such that the guide rod 112 and the bracket 130 do not move relative to one another, for instance by a friction fit between the guide rod 112 and the inner bore of the bracket 130. In another embodiment the bracket 130 can include a set screw or other fastener configured to be received within a recess of the bracket 130 and tightened against the guide rod 112 to secure the guide rod 112 relative to the bracket 130. The housing 104 and probe 102 can translate along the guide rod 112 from the first contracted configuration (as shown in FIG. 8A) to the second expanded configuration (as shown in FIG. 8B). In the first contracted configuration, the free end 108 of the probe 102 is removed from the lobes 46 of the collection body 44, such that the collection body 44 is free to rotate about the axis of rotation 22 without interference from the probe 102.

As described in detail above, separation of a multiple component sample (lysed BMA 11) into its separate components (cell pellet 15 and supernatant 13) can be performed by rotation of the bowl portion 24 and the lid 70 about the axis of rotation 22. As shown, the desired component (cell pellet 15) is concentrated within the pocket 62 near the apex 50 of the lobes 46 of the collection body 44. The collector 100 can then be moved into the second expanded configuration such that the free end 108 of the probe 102 is positioned within the desired component, for instance cell pellet 15, of the multiple component sample. The collector 100 can then collect a sample of the cell pellet 15 or other desired component.

In one embodiment the bracket 130 can be secured to the housing 300 such that bracket 130 and the secured collector 100 are in a fixed radial position relative to the separator 20. Thus to collect the desired component from a first of the lobes 46, the collection body 44 can be rotated about the axis of rotation 22 until a reference point of the collector 100, for example the guide rod 112 is aligned with the apex 50 of one of the lobes 46. In another embodiment, the reference point can be the free end 108 of the probe 102. The collector 100 can then be transitioned from the first retracted configuration to the second expanded configuration enabling the probe 102 to collect a sample of the desired component of the multiple component sample. During the transition from the first retracted configuration to the second expanded configuration the housing 104 translates along the guide rod 112 in a direction toward the apex 50 of the lobe 46 with which the collector 100 has been aligned. The housing 104 is translated until the free end 108, and the cannulation 110, of the probe 102 is positioned within the cell pellet 15.

The collector 100 can then be actuated, for example by moving the plunger 128 to create a negative pressure within the cannulation 110, which is pneumatically connected to the syringe 118. The negative pressure within the cannulation 110 draws the cell pellet 15 into the free end 108 of the probe 102 and through the passage 122 until the cell pellet is deposited within the receiving chamber 119 of the syringe 118. Once the desired component has been collected from the lobe 46, the collector 100 is transitioned back into the first retracted configuration. Then the collection body 44 can be rotated again until the probe 102 is aligned with the apex 50 of another lobe 46. The process described above can then be repeated until the desired component has been collected from each of the lobes 46.

Referring to FIGS. 8C and 8D, in another embodiment, the bracket 130 can be attached to the lid 70 and positioned at least partially within one of the openings 82 such that the bracket 130 can move relative to the lid 70, for instance such that the bracket 130 can rotate about the axis of rotation 22 relative to the lid 70. As shown in the illustrated embodiment, the dome portion 78 can include a lip 94 that defines the opening 82. The bracket 130 includes a recess 132 that is configured to slidably receive the lip 94 such that the bracket 130 can move relative to the lid 70, for instance by sliding the lip 94 within the recess 132 to rotate the bracket 130 about the axis of rotation 22 relative to the lid 70. In one embodiment the lip 94 and the recess 132 can include a tongue and groove connection. In another embodiment the bracket 130 can be rotationally locked to the lid 70 during rotation of the separator 20 about the axis of rotation 22 and then unlocked after rotation about the axis of rotation 22 has been completed.

The moveable bracket 130 relative to the lid 70 enables a reference point of the collector 100, for example the guide rod 112 or the probe 102, to be aligned with the apex 50 of one of the lobes 46. The collector 100 can be transitioned from the first retracted configuration into the second expanded configuration such that the free end 108 of the probe 102 is disposed within the desired component. After a concentrated sample of the desired component has been collected (as described above) the collector 100 can be transitioned back into the first retracted configuration. The bracket 130 and the attached collector 100 can then translate along the lip 94 of the opening 82 such that the collector 100 rotates about the axis of rotation 22 relative to the lid 70 and the bowl portion 24 until the collector 100 is aligned with the apex 50 of another lobe 46. The process can then be repeated until a concentrated sample of the desired component has been collected from each lobe 46.

Referring to FIG. 9, in accordance with another embodiment the collector 100 can include a syringe 218 with a probe 220. The syringe 218 can be attached to the housing 300 or the lid 70 as described above, or can be separate from the housing 300 and the lid 70, for instance such that the syringe 218 is held by hand by a user of the device 18. The probe 220 can be straight or as shown in the illustrated embodiment, can be bent at an angle configured to allow the probe 220 to pass through an opening 82 in the lid 70 and into the separated desired component, for instance cell pellet 15, located in the pocket 62 near the apex 50. The bent probe 220 allows a non-direct approach to the pocket 62 of the lobe 46. A non-direct approach can be appropriate if the direct approach is blocked by the structure of the separator 20, for instance by a locking cap 222 or other securing mechanism that extends through the lid 70 in a direction parallel to the axis of rotation 22.

Referring to FIG. 10, in one embodiment, after centrifugation of the multiple component sample, the desired component, for example the cell pellet 15, is separated from the remaining component, for example the supernatant 13, such that the cell pellet 15 is positioned within the pocket 62 adjacent to the apex 50. As shown in the illustrated embodiment, substantially the entire cell pellet 15 is separated from the supernatant 13, such that the cell pellet 15 is positioned within the pocket 62, adjacent the apex 50, and radially outward from the supernatant 13.

According to one embodiment, the collector 100 includes a housing 104 that is movably attached to a guide rod 112. The collector 100 further includes a probe 102 that is supported by the housing 104, such that the probe 102 is configured to collect the cell pellet 15. The collector 100 can further include a scraper 120 that is supported by the housing 104. In one embodiment, the probe 102 and the scraper 120 are each attached on opposite sides of the housing 104, for example the probe 102 and the scraper 120 can be attached to the flanges 121 of the housing 104. The probe 102 and the scraper 120 are each secured relative to the housing 104 such that the probe 102 and the scraper 120 each translate along with the housing 104 as the collector 100 is transitioned from the first contracted configuration to the second expanded configuration.

As shown in the illustrated embodiment, the scraper 120 includes an attached end 134 that can be secured to the housing 104, a free end 136 opposite the attached end 134, and a scraper body 138 that extends from the attached end 134 to the free end 136 along a central scraper axis 140. In one embodiment, the central scraper axis 140 can be curved as shown. In another embodiment, the central scraper axis 140 can be substantially straight. Similarly, the probe 102, in one embodiment can extend from the attached end 106 to the free end 108 along a central probe axis 141. In one embodiment, the central probe axis 141 can be curved as shown. In another embodiment, the central probe axis 141 can be substantially straight. In another embodiment, the collector 100 can include the probe 102 that is configured to collect substantially the entire cell pellet 15 without the inclusion of the scraper 120.

In use, the collector 100 is configured to be aligned with one of the lobes 46, for example such that the guide rod 112 is aligned with the apex 50. Once the collector 100 is aligned with the lobe 46 the collector 100 is transitioned from the first retracted configuration to the second expanded configuration, the probe 102 translates with the housing 104 along the guide rod 112 in a direction, for example radially or specifically in the longitudinal direction L, toward the apex 50. As the housing 104 and the probe 102 translate in the radial direction toward the apex 50, the free end 108 of the probe 102 can, in one embodiment, be advanced into the lobe 46 until the free end 108 is positioned within the supernatant 13. The collector 100 can then be actuated, as described in greater detail below, to remove a portion, for example substantially all, of the supernatant 13 from the lobe 46. In one embodiment, the removal of the supernatant 13 can be repeated for all of the lobes 46 of the collection tray 26.

The free end 108 of the probe 102 can then be advanced further in the radial direction until the free end 108 is positioned within the pocket 62 and within the cell pellet 15. The collector 100 can then be actuated, as described in greater detail below, to remove a portion, for example substantially the entirety of the cell pellet 15 from the lobe 46. In one embodiment, the removal of the cell pellet 15 can be repeated for all of the lobes 46 of the collection tray 26. In another embodiment, the free end of the probe 102 can be advanced through the supernatant 13 and into the cell pellet 15 without withdrawing the supernatant 13.

Referring to FIGS. 11A to 11D, in another embodiment, after centrifugation, the desired component, for example the cell pellet 15, is separated from the supernatant 13 such that a portion of the cell pellet 15 is disposed within the pocket 62 adjacent the apex 50 of the lobe 46, and another portion of the cell pellet 15′ may be disposed along the side wall 52 in a thin layer. In one embodiment, as shown in FIG. 10 above, the separator 20 can be configured such that after centrifugation a minimal amount, or no amount, of the cell pellet 15′ will be disposed along the side wall 52, and instead nearly the entire cell pellet 15 will be collected within the pocket 62 adjacent the apex 50. In another embodiment, for example if the desired component, such as the cell pellet 15, is sticky, a portion of the cell pellet 15′ may collect along the lobe side walls 52 after centrifugation. The collector 100 can include at least one scraper 120 to aid in the collection of the cell pellet 15 and 15′. The scraper 120 is configured to aid in the collection of a concentrated sample of the desired component as described in further detail below.

In one embodiment, the collector 100 includes a probe 102, for example the probe/scraper body 103, and a scraper 120 that are each supported by the housing 104 of the collector 100. In one embodiment, the probe 102 and the scraper 120 are each attached on opposite sides of the housing 104, for example the probe 102 and the scraper 120 can be attached to the flanges 121 of the housing 104. The probe 102 and the scraper 120 are each secured relative to the housing 104 such that the probe 102 and the scraper 120 each translate along with the housing 104 as the collector 100 is transitioned from the first contracted configuration to the second expanded configuration. As shown in the illustrated embodiment, the scraper 120 includes an attached end 134 that can be secured to the housing 104 as shown, a free end 136 opposite the attached end 134, and a scraper body 138 that extends from the attached end 134 to the free end 136 along a central scraper axis 140.

In one embodiment, the central scraper axis 140 can be curved as shown. Similarly, the probe 102, in one embodiment can extend from the attached end 106 to the free end 108 along a central probe axis 141. The scraper body 138 defines a length measured from the attached end 134 to the free end 136 along the central scraper axis 140. The scraper body 138 can further include a tip portion 142 that is configured to aid in the collection of a concentrated sample of a desired component of a multiple component sample.

In use, as the collector 100 is transitioned from the first retracted configuration to the second expanded configuration, the probe 102 and the scraper 120 each translate with the housing 104 along the guide rod 112 in a direction, for example radially or specifically in the longitudinal direction L, toward the apex 50. As the housing 104, the probe 102 and the scraper 120 translate in the direction toward the apex 50, the tip portion 142 of the scraper 120 and the free end 108 of the probe 102 each move into contact one of the side walls 52 near the base portion 48 (as shown in FIG. 11B).

As the collector 100 continues to transition from the first retracted configuration to the second expanded configuration, and the probe 102 and the scraper 120 continue to advance in the radial direction toward the apex 50, the tip portion 142 of the scraper 120 and the free end 108 of the probe 102 each translate along the side wall 52 gathering and moving the additional portion of the cell pellet 15′ toward the portion of the cell pellet 15 in the pocket 62 adjacent the apex 50 (as shown in FIG. 11C). In one embodiment, at least one of the probe 102 and scraper 120 can be constructed of a flexible material such as a plastic, or a polymer. As the collector 100 transitions from the first retracted configuration to the second expanded configuration, the probe 102 and the scraper 120 abut the side wall 52 and flex such that the curvature of the central probe axis 141 and the central scraper axis 140 increases. In another embodiment, at least one of the probe 102 and the scraper 120 can be constructed of a substantially rigid material and flexibly or rotatably connected, for example hinged, to the housing 104. In another embodiment, the probe 102 can be supported by the housing 104 such that the probe 102 is substantially aligned with the guide rod 112 (as shown in FIG. 7D), and therefore does not need to flex as the collector 100 transitions from the first retracted configuration to the second expanded configuration.

Once the collector 100 has fully transitioned into the second expanded configuration (as shown in FIG. 11D), the tip portion 142 of the scraper 120 and the free end 108 of the probe 102 have gathered the cell pellet 15 into a single location within the pocket 62. The collector can include a stop 143, for example supported by the guide rod 112, configured to prevent further translation of the housing 104 in the direction toward the apex 50. For example, the stop 143 can include a projection, attached to the guide rod 112 that abuts the housing 104 once the collector 100 is in the second expanded configuration. As shown, in the second expanded configuration, the free end 108 of the probe 102 is positioned within the cell pellet 15 such that cell pellet 15 can be drawn into the probe 102 and gathered for collection.

Referring to FIGS. 7B and 11C, when the collector 100 is in the second expanded configuration with a collection container inserted at the attachment point 125 of the housing 104 and the free end 108 of the probe 102 positioned within the cell pellet 15, a concentrated sample of the desired component, for example the cell pellet 15, can be collected. A negative pressure is created within the passage 122, for example by pulling back on the plunger 128 of the syringe 118 in a direction away from the attachment point 125. The negative pressure within the passage 122, including the cannula 110, draws the cell pellet 15 located near the free end 108 of the probe 102 into the cannulation 110 of the probe 102. The collected cell pellet 15 travels along the passage 122 through the cannulation 110 from the free end 108 to the attached end 106. The collected cell pellet 15 can then travel through the tube 123 that is pneumatically connected to the cannulation 110 of the probe 102. The collected cell pellet 15 can then travel, either directly or via a continuation of the passage 122 within the housing 104, to the attachment point 125 and into the receiving chamber 119 of the syringe 118.

Referring to FIG. 12, the device 18 can include a housing 300 that is configured to at least partially enclose the separator 20 and the collector 100. The housing can be further configured to sit on a table top, for instance in an operating room. The housing can be sized such that the device 18 is easily portable and disposable after use.

The housing 300 includes a top surface 302, a bottom surface 304, and a housing body 306 that extends from the top surface 302 to the bottom surface 304. The housing body 306 can include a base portion 308 and a cap portion 310. The base portion 308 defines an inner cavity 312 that is configured to enclose the separator 20. The separator 20 can be mounted within the inner cavity 312 such that the separator can rotate without interference from the housing body 306. The inner cavity 312 can additionally enclose a motor 400 and a drive shaft 402 rotationally coupled to the motor 400. The drive shaft 402 can be rotationally coupled to the recess 37 of the engagement mechanism 33 of the separator 20 such that the motor 400 can provide a rotational force to the separator 20 that causes the separator to rotate about the axis of rotation 22.

The base portion 308 can further include a window 314 (or other opening) such that an operator of the device 18 can see the separator 20. The window 314 can be configured such that the pocket 62 of the separator is visible through the window 314 allowing for visualization of the pocket 62 during alignment of the pocket 62 with the collector 100 and collection of the desired component from the pocket 62 by the collector 100. Additionally, the device 18 can include a power supply, for example batteries, to power any electrical components of the device 18. The device 18 can further include a printed circuit board that is configured to support and connect electronic components of the device 18 and provide various logic functions. One or more LEDs 320 can be included to indicate the status of the device 18 (e.g., ready to centrifuge, centrifuging, centrifuging complete and ready for collection).

The cap portion 310 is configured to be secured to the base portion 308 to at least partially enclose the separator 20 and the collector 100. During rotation of the separator 20 about the axis of rotation 22, the cap portion 310 can prevent an operator of the device 18 from touching any moving parts of the device 18 during the centrifugation process. In one embodiment, the device 18 includes a cap sensor switch and linkage configured to detect if the cap portion 310 is correctly in place relative to the base portion 308, and allow the motor 400 to spin only if the cap portion 310 is correctly in place relative to the base portion 308. After rotation of the separator 20 has completed and during collection of the desired component, the cap portion 310 can be removed from the base portion 308 such that access to the collector 100 is provided to an operator of the device 18. The housing body 306 can further include a ledge 316 that is positioned between the base portion 308 and the cap portion 310. The ledge 316 is configured to receive the bracket 130 such that the collector 100 is positioned relative to the separator 20 such that when the collector 100 is in the first retracted configuration (as shown in FIG. 12) the separator 20 is free to rotate about the axis of rotation 22, and when the collector 100 is in the second expanded configuration the probe 102 is disposed within the pocket 62 to collect a sample of the desired component.

Referring to FIGS. 1B to 12, the device 18 can be used in a process to harvest, separate, concentrate, and collect an amount of a desired component of a multiple component sample. A volume, for instance between about 8 cc and about 50 cc, of a multiple component sample (such as withdrawn BMA 1) can be harvested from a bone, for example by puncturing the bone with a needle, for example that is connected to a syringe, and drawing an amount of the withdrawn BMA 1 into the syringe. The harvested BMA 1 can then be placed in the bowl portion 24 of the separator 20 of the device 18. A volume, for instance between about 16 cc and about 100 cc, of lysing agent can then be added to the withdrawn BMA 1 which results in a sample of lysed BMA 11. The lysed BMA 11 contains a desired component (such as the cell pellet 15) and a remaining portion (such as the supernatant 13). The cell pellet 15 can then be separated from the supernatant 13 and then collected by the device 18.

In use, the separator 20 containing the lysed BMA 11 can rotate around the axis of rotation 22 at a desired angular velocity for a desired amount of time, for example 3000 RPMs (or about 500 G's) for about 5 minutes, such that the cell pellet 15 will ride up the bowl wall 38 (due to the bowl angle θ as described above), over the upper lip 36 and into the collection tray 26. As the separator 20 continues to rotate about the axis of rotation 22 the cell pellet 15 will pass into the basin 57 of the lobe 46 and move radially away from the axis of rotation 22 and collect in the pocket 62. The cell pellet 15 can then be collected from the pocket 62 of each of the lobes 46 by the collector 100.

In one embodiment, if a relatively smaller volume of lysed BMA 11 has been centrifuged, the resulting cell pellet 15 may only fill a portion of the pocket 62. The collector 100 can be transitioned to a third intermediate configuration in which the collector 100 is partially transitioned from the first retracted configuration to the second expanded configuration. In the third intermediate configuration the free end 108 of the probe 102 is positioned within the remaining component and close to, but not within the desired component, for example the cell pellet 15. In one embodiment the third intermediate position is determined visually, through the window 314 in the base portion 308. In another embodiment, the collector 100 can include a series of markings 127, for example on the guide rod 112, such that when the housing 104 is aligned with the appropriate marking 127 (based on the initial volume of BMA), the collector 100 is in the third intermediate configuration.

A waste syringe 118 can be connected to the attachment point 125 and the collector 100 can be actuated such that the remaining component is removed from the pocket 62 and drawn into the waste syringe 118. Once the remaining component has been removed from the pocket 62, the waste syringe 118 can be removed from the attachment point 125 and replaced by a second syringe 118. In another embodiment, once the remaining component has been removed from the pocket 62, the collector 100 can be transitioned into the first retracted configuration. The collector 100 can then be aligned with another of the lobes 46 and the remaining steps above repeated until the remaining component has been removed from all of the lobes 46. The waste syringe 118 can be removed from the attachment point 125 and replaced by a second syringe 118.

The collector 100 can then be fully transitioned into the second expanded configuration such that the free end 108 of the probe 102 is disposed within the desire component. The collector 100 can then be actuated to draw the desired component into the second syringe 118 for collection. The collector can then be transitioned back into the first retracted configuration and the second syringe 118 can be removed from the attachment point 125. This process can then be repeated as needed for the remaining lobes 46.

In another embodiment, if a relatively larger volume of lysed BMA 11 has been centrifuged, the resulting cell pellet 15 may substantially fill the pocket 62. In this case, a syringe 118 can be attached to the attachment point 125, the collector 100 can be transitioned from the first retracted configuration to the second expanded configuration, the collector 100 can be actuated to create a negative pressure within the passage 122, drawing the desired component into the probe 102 through the passage 122 and into the syringe 118. The collector 100 can then be transitioned from the second expanded configuration to the first retracted configuration. The collector 100 can then be aligned with the apex 50 of another lobe 46, and the process repeated as needed for any remaining lobes 46.

In one embodiment, the collector can be used to remove at least a portion of the supernatant 13 from the basin 57 of each of the lobes 46. Then the collector can also be transitioned from the first retracted configuration to the second expanded configuration causing the scrapers 120 of the collector 100 to ride along the inner side walls 53 of each of the lobes 46 to gather the cell pellet 15 in each of the pockets 62. The collector 100 is then transitioned from the second expanded configuration to the first retracted configuration before the separator 20 is again rotated about the axis of rotation 22 to concentrate the cell pellet 15 in the pockets 62 at the most radially distant location within the lobes 46. The collector 100 is then again transitioned to the second expanded configuration and a sample of the cell pellet 15 is collected from the pocket 62 of each of the lobes 46. If any cell pellet 15 remains in the lobes 46 the rotation and collection steps can be repeated as desired.

In another embodiment, a solution that loosens the cell pellet 15 from the inner side walls 53 of the lobes can be used between rotation cycles to increase the amount of cell pellet 15 gathered during each collection phase. Once the desired amount of cell pellet 15 has been collected the device 18 can either be disposed of or broken down and sterilized for re-use.

It will be appreciated by those skilled in the art that changes could be made to the embodiments described above without departing from the broad inventive concept thereof. It is understood, therefore, that this disclosure is not limited to the particular embodiments disclosed, but it is intended to cover modifications within the spirit and scope of the present disclosure as defined by the claims.

What is claimed:

1. A collection tray configured to rotate about an axis of rotation to separate a multiple component sample into a desired component and a remaining component, the collection tray defining a ray line that extends perpendicularly from the axis of rotation, the collection tray comprising:
a collection body configured to receive the multiple component sample;
a plurality of lobes supported by the collection body, each of the lobes having two lobe base portions, an apex, and two lobe side walls that each extend between one of the lobe base portions and the apex, wherein each of the lobes defines a straight lobe line that perpendicularly intersects one of the lobe side walls at a point located radially between the respective lobe base portion and the apex, such that the ray line intersects the point so as to define a lobe angle measured between the ray line and the lobe line;
wherein the lobe angle is greater than a specific angle, such that the arctangent of the specific angle is equal to the effective coefficient of friction of the desired component and the lobe side wall.

2. The collection tray of claim 1, wherein the lobe side wall defines an inner surface and an opposed outer surface, and the inner surface presents a curvature both proximal to the point and distal to the point.

3. The collection tray of claim 1, wherein the lobe side walls include an inner side wall, an opposed outer side wall and a midpoint located radially halfway between the respective lobe base portion and the apex, the lobe side walls each include a proximal portion located between the lobe base portion and the midpoint and the proximal portion is curved such that no portion of the inner side wall extends parallel to the radial ray.

4. The collection tray of claim 1, wherein each of the lobes defines a straight lobe line that perpendicularly intersects one of the lobe side walls at any point located radially between the respective lobe base portion and the apex, such that the ray line intersects the point so as to define a lobe angle measured between the ray line and the lobe line.

5. The collection tray of claim 1, wherein the multiple component sample is BMA with lysed red blood cells and lysing agent, and the desired component is a cell pellet containing stem cells.

6. The collection tray of claim 1, wherein each of the lobe base portions is located radially closer to the axis of rotation than the respective apex.

7. The collection tray of claim 1, wherein the lobe angle is between about 10 and about 40 degrees.

8. The collection tray of claim 7, wherein the lobe angle is about 20 degrees.

9. The collection tray of claim 8, wherein the plurality of lobes includes four lobes.

10. A device configured to separate a multiple component sample into a desired component and a remaining component, the device comprising:
a bowl portion defining an interior configured to receive the multiple component sample, the bowl portion configured to rotate about an axis of rotation; and
a collection tray configured to be supported by the bowl portion so as to rotate about the axis of rotation, the collection tray defining a ray line that extends perpendicularly from the axis of rotation, the collection tray including at least one lobe that has two lobe base portions, an apex, and two lobe side walls that each extend from one of the lobe base portions to the apex, wherein the at least one lobe at least partially defines a basin that is in fluid communication with the interior of the bowl portion such that the multiple component sample is transferable from the interior to the basin during rotation of the bowl portion about the axis of rotation,
wherein the at least one lobe further defines a lobe line that is different from the ray line, the ray line intersects one of the lobe side walls at a point along the lobe side wall, and the lobe line perpendicularly intersects the point so as to define a lobe angle between the ray line and the lobe line.

11. The device of claim 10, wherein the lobe angle is greater than a specific angle, such that the arctangent of the specific angle is equal to the effective coefficient of friction of the desired component and the lobe side wall.

12. The device of claim 11, wherein the rotation of the collection tray about the axis of rotation causes the desired component to accumulate adjacent to the apex of each of the at least one lobe.

13. The device of claim 10, wherein the lobe angle is between about 10 and about 40 degrees.

14. The device of claim 13, wherein the lobe angle is about 20 degrees.

15. The device of claim 14, wherein the at least one lobe includes at least two lobes.

16. The device of claim 15, wherein the at least two lobes includes four lobes.

17. The device of claim 16, wherein the bowl portion further includes a bowl bottom and a bowl wall extending out from the bowl bottom, the bowl portion including a bowl angle measured between an intersecting radial ray that extends perpendicular to the axis of rotation and a bowl line that is normal to the bowl wall at the intersection, the bowl angle being greater than a specific bowl angle such that the arctangent of the specific bowl angle is equal to the effective coefficient of friction of the desired component and the bowl wall.

18. The device of claim 17, wherein the bowl angle is about 20 degrees.

19. The device of claim 10, wherein the bowl portion further includes a bowl bottom and a bowl wall extending out from the bowl bottom, the bowl portion including a bowl angle measured between an intersecting radial ray that extends perpendicular to the axis of rotation and a bowl line that is normal to the bowl wall at the intersection, the bowl angle being greater than a specific bowl angle such that the arctangent of the specific bowl angle is equal to the effective coefficient of friction of the desired component and the bowl wall.

20. The device of claim 19, wherein the bowl angle is about 20 degrees.

21. The device of claim 10, wherein the multiple component sample is withdrawn BMA plus a lysing agent, and the desired component is a cell pellet containing stem cells.

22. The device of claim 10, wherein the ray line intersects the one of the lobe side walls at any point along the lobe side wall, and the lobe line perpendicularly intersects the point so as to define the lobe angle between the ray line and the lobe line.

23. A process to process a withdrawn BMA sample, the process including the steps of:
combining the withdrawn BMA sample and a red blood cell lysing agent so as to form a multiple component sample;
rotating a device about an axis of rotation, the device containing the multiple component sample, so as to separate the multiple component sample into a desired component and a remaining component; and
collecting at least a portion of the desired component.

24. The process of claim 23, further comprising the step of collecting at least a portion of the remaining component prior to the step of collecting at least a portion of the desired component

25. The process of claim 23, wherein the multiple component sample is BMA with lysed red blood cells and lysing agent and the desired component is a cell pellet containing stem cells.

26. The process of claim 23, further comprising before the combining step:
inserting the withdrawn BMA sample into a bowl portion of the device.

27. The process of claim 23, further comprising after the combining step:
inserting the multiple component sample into a bowl portion of the device.

28. The process of claim 23, further comprising;
providing a collection tray configured to be secured to the bowl portion such that the bowl portion and collection tray are rotationally secured relative to one another, the collection tray includes at least two lobes, the at least two lobes each having two lobe base portions, a apex, and two lobe side walls, each of the lobe side walls extends from one of the lobe base portions to the apex;
wherein during the rotating step, the desired component gathers near the apex of each of the at least two lobes.

29. The process of claim 28, wherein the providing step further comprises, a ray line that extends radially from and perpendicular to the axis of rotation, the ray line intersects one of the lobe side walls at a point and, a lobe line that perpendicularly intersects one of the lobe side walls at the point such that a lobe angle is defined between the ray line and the lobe line.

30. The process of claim 28, wherein the lobe angle is between about 10 and about 40 degrees.
